(19) United States
(12) Patent Application Publication
(10) Pub. No.: US 2011/0104250 A1
(43) Pub. Date: May 5, 2011

(54) Redox-gated Liposomes

(76) Inventors: Robin L. McCarley, Prairieville, LA (US); Winston Z. Ong, Stoneham, WA (US)

(21) Appl. No.: 12/676,726

(22) PCT Filed: Sep. 8, 2008
(86) PCT No.: PCT/US08/10496

§ 371 Date: Jan. 18, 2011

Related U.S. Application Data
(60) Provisional application No. 60/970,912, filed on Sep. 7, 2007.

(51) Int. Cl. A61K 9/127; C07F 9/02; C12N 5/02

(52) U.S. Cl. 424/450; 554/84; 435/375

(57) ABSTRACT

The present invention provides lipid compounds for delivering therapeutic amounts of active agents in response to enzymatic activities of cancer tissues. Lipid compositions may include one or more drugs, or a biologically-active agent, encapsulated within liposomes.

### CROSS-REFERENCE TO RELATED APPLICATIONS

This application claims the benefit of U.S. patent application Ser. No. 60/970,912, filed Sep. 7, 2007, which is hereby incorporated by reference in its entirety.

### STATEMENT REGARDING FEDERALLY SPONSORED RESEARCH OR DEVELOPMENT

The invention was made with U.S. Government support under contract no. CHE-0108961 awarded by the National Science Foundation. The U.S. Government has certain rights in the invention.

### BACKGROUND OF THE INVENTION

1. Field of the Invention

The present invention relates generally to drug, bio-affecting, and body treating compositions that possesses some physical form, or whose components are associated as plural layers or parts. The present invention relates more specifically to such compositions in which the physical form is a pharmaceutical substance contained in a lipid bilayer.

2. Description of Related Art

Liposomes consist of at least one lipid bilayer membrane enclosing an aqueous internal compartment. They may be characterized by membrane type and by size, and are also referred to as vesicles. Small unilamellar vesicles (SUVs) possess a single bilayer membrane, and typically range between 0.02 and 0.05 μm in diameter. Large unilamellar vesicles (LUVs) are typically larger than 0.05 μm. Oligolamellar large vesicles and multilamellar large vesicles have multiple (usually concentric) membrane layers, and are typically larger than 0.1 μm. Liposomes with several non-concentric membranes (i.e., smaller vesicles contained within a larger vesicle) are called multilamellar vesicles.

Conventional liposomes may be formulated to carry drugs or other active agents within either the aqueous interior space (water-soluble drugs) or the lipid bilayer (water-insoluble drugs). Biologically active agents with short half-lives in the bloodstream are particularly well-suited to delivery via liposomes because the agents are isolated within liposomal membranes, thus preventing or slowing their degradation. Many anti-neoplastic agents, for example, are known to have a short half-life in the bloodstream such that their parenteral use is not feasible. Despite the promising potential of liposomal drug delivery systems, their use for site-specific delivery of active agents via the bloodstream is severely limited because they are rapidly cleared from the blood by cells of the reticuloendothelial system. Consequently, anti-neoplastic agents delivered using conventional liposomes may fail to destroy the targeted neoplastic cells and still produce the undesirable side-effects that are the hallmark of chemotherapy.

Stimulus-responsive liposomes have emerged as promising drug carriers due to the inherent advantages associated with liposomal formulation and the controllable release of liposomal cargo (Huang Z. & Szoka F. C. “Bioresponsive Liposomes and Their Use for Macromolecular Delivery” in: Liposome Technology, pp. 165-96 (Gregory Gregoriadis ed., CRC Press 2006)). The lipids in these liposomes generally contain a stimulus-responsive subunit that is responsible for gating the stability and/or permeability of the lipid bilayer. These liposomes are sometimes referred as “smart” delivery systems because unloading of the encapsulated payload requires a stimulus. Ideally, a stimulus triggers the onset of cargo unloading, thereby allowing the carrier-cargo ensemble to be constructed without prematurely sacrificing or exposing the encapsulated cargo to the external environment. Various physiological environments (such as low endosomal pH or elevated enzymatic activity) or external sources (including radiation and hyperthermia) may supply the stimulus necessary to induce unloading of the liposomal cargo, either by perturbing liposome permeability or by completely disrupting the noncovalent stability of the bilayer assembly. For instance, it has been shown that the permeability of pH- and radiation-sensitive liposomes can be perturbed by the acid-triggered depegylation of PEG-conjugated lipids and photochemical “uncorking” of o-benzyl-protected lipids, respectively, leading to the release of the encapsulated payload.

It is desirable to devise liposome formulations capable of delivering therapeutic amounts of active agents in response to enzymatic activities of cancer tissues.

### BRIEF SUMMARY OF THE INVENTION

In one aspect, the present invention includes novel lipids of Formula 1:

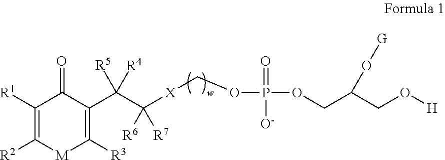

wherein:
R¹, R², R³, R⁴, R⁵, R⁶, R⁷, and R⁸ represent, independently, H, Cl, Br, I, CH₃, n-C_{y}H_{2y+1} (where y is an integer value from 1 to 3), n-CⱼH₂ⱼ₊₁O (where j is an integer value from 1 to 3), or (EO)_{z}—R⁹ (where EO is ethylene oxide and z is an integer value from 3 to 100);
R⁹ is H, CH₃ (“methyl”), or CF₃CH₂OC(O)CH₂;
M is CH₂, —C(O)— (“carbonyl”), or CH—R⁸;
X is —C(O)NH— (“C-amide”), —C(S)NH— (“C-thioamide”), —C(O)O— (“C-ester”), —C(O)S— (“C-thioester”), —C(O)NHC(O)— (“imide”), —C(O)OC(O)— (“anhydride”), —CH₂OC(O)— (“O-ester”), —CH₂SC(O)— (“S-thioester”), —CH₂NHC(O)— (“N-amide”), —CH₂NHC(S)— (“N-thioamide”), —CH₂OC(O)O— (“carbonate”), —CH₂NHC(O)NH— (“urea”), —CH₂NHC(S)NH— (“thiourea”), —CH₂OC(O)— (“O-ester”), —CH₂OC(O)NH— (“O-carbamate”), —CH₂NHC(O)O— (“N-carbamate”), —CH₂NHC(O)S— (“N-thiocarbamate”), —CH₂SC(O)NH— (“S-thiocarbamate”), —CH₂OS(O)(O)— (“mesylate”), or —CH₂OP(O)(O)O— (“phosphate”); and
G and H are, independently, oleoyl, elaidoyl, linoeoyl, linolenoyl, or —C(O)[n-CₜH₂ₜ₊₁] (where t is an integer value from 6 to 18); and
w is an integer value between 1 and 2, indicating the number of methylenes (—CH₂—).

Preferred are those compounds wherein R¹, R², R³, R⁴, and R⁵ are CH₃, R⁶ and R⁷ are H, M is —C(O)—, X is —C(O)NH—, and both G and H are elaidoyl, and w is 1 or 2.

More preferred are those compounds wherein R¹, R², R³, R⁴, and R⁵ are CH₃, R⁶ and R⁷ are H, M is —C(O)—, X is —C(O)NH—, and both G and H are linoeoyl, and w is 1 or 2.

Even more preferred are those compounds wherein R¹, R², R³, R⁴, and R⁵ are CH₃, R⁶ and R⁷ are H, M is —C(O)—, X is —C(O)NH—, both G and H are linolenoyl, and w is 1 or 2.

Particularly preferred are those compounds wherein R¹, R², R³, R⁴, and R⁵ are CH₃, R⁶ and R⁷ are H, M is —C(O)—, X is —C(O)NH—, and both G and H are oleoyl, and w is 1 or 2.

In another aspect, the present invention includes liposomes comprising the above-described lipids of Formula I. Preferred liposome compositions include the preferred lipids described above. In constructing the liposomes, various mixtures of the lipids of Formula I can be used in combination with one another. More preferred lipid compositions are those wherein one or more drugs or a biologically-active agents is encapsulated within the liposomes.

In another aspect, the present invention includes methods for delivering one or more drugs or biologically-active agents to cells, comprising encapsulating the agent in a liposome comprising the above-described lipids of Formula I to form a liposome-bioactive complex and contacting the cells with the complex. In this aspect, the one or more drugs or biologically-active agents (sometimes referred to herein as “bioactive agents”) may be, without limitation, antitumor agents, antibiotics, anthracycline antibiotics, immunodilators, anti-inflammatory drugs, drugs acting on the central nervous system, proteins, peptides, doxorubicin, daunorubicin, epirubicin, idarubicin, and mitoxantrone.

In another aspect, the present invention includes methods of treating a disease in a patient comprising administering to the patient a therapeutically effective amount of a pharmaceutical composition containing one or more drugs or bioactive agents encapsulated in a liposome comprising lipids of Formula I. In this aspect of the invention, the present invention includes a pharmaceutical formulation comprising the liposome comprising lipids of Formula I, and a physiologically-acceptable adjuvant thereof. In this aspect, the one or more drugs or biologically-active agents may be, without limitation, antitumor agents, antibiotics, anthracycline antibiotics, immunodilators, anti-inflammatory drugs, drugs acting on the central nervous system, proteins, peptides, doxorubicin, daunorubicin, epirubicin, idarubicin, and mitoxantrone.

In still another aspect, the present invention includes methods for delivering therapeutic agents such as drugs, vaccines, and various other biologically-active agents to a patient in need thereof, comprising administering to the patient a therapeutically effective amount of such biologically-active agent in a liposome of the invention. In this aspect, the one or more drugs or biologically-active agents may be, without limitation, antitumor agents, antibiotics, anthracycline antibiotics, immunodilators, anti-inflammatory drugs, drugs acting on the central nervous system, proteins, peptides, doxorubicin, daunorubicin, epirubicin, idarubicin, and mitoxantrone.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 shows the structures of DOPE-quinones 1-Q1 and 1-Q3, the corresponding model compounds 2-Q1 and 2-Q3, and lactones 3-HQ3 and 3-HQ1.

FIG. 2 depicts the characterization of calcein-free liposomes. FIG. 2A is a cryo-TEM micrograph of 1-Q1, and FIG. 2B shows the size distribution by DLS intensity of 1-Q3 at 25° C.

FIG. 3 shows the time-course dependence of the emission intensities (I) of calcein-loaded liposomes at 515 nm (λₑₓ=495 nm) under ambient conditions. Solid (A and B) and perforated (C-E) traces represent the liposomes of 1-Q3 and 1-Q1, respectively. Square symbols represent the addition of 100 equivalents of Na₂S₂O₄ (□ for traces A and C at 125 min and trace E at 1260 min) and/or 0.1% w/v Triton® X-100 (▪ for traces B-E) to the liposomal solutions.

FIG. 4 shows a mechanism whereby reductive lactonization of 1-Q3 produces 3-HQ3 and DOPE.

FIG. 5 shows a proposed mechanism for the conversion of liposomal 1-Q3 from unilamellar (L_{α}) liposomes to inverted hexagonal (H_{II}) micelles. Step A: Na₂S₂O₄ reduces the 1-Q3 lipids that are located in the outer layer of the liposome to 1-HQ3. Step B: 1-HQ3 dissociates into lactone 3-HQ3 and DOPE, as described in FIG. 4. Step C: migration of HQ3 and DOPE create voids in the bilayer, thereby releasing the encapsulated payload (indicated herein as calcein dye).

FIG. 6 is a schematic diagram showing the synthesis of DOPE quinones 1-Q1 and 1-Q3, and the corresponding model compounds 2-Q1 and 2-Q3.

FIG. 7 shows the time-dependent release of 3-HQ from 2-HQ3 in 0.1 M phosphate buffer (pD=7). Line shows the best fit to the experimental data.

FIG. 8 shows cyclic voltammograms of 2-Q1 (1.0×10⁻³ M) and 2-Q3 (1.0×10⁻³ M) in 0.1 M phosphate buffer, pH 7.1, at ambient temperature using a glassy carbon electrode (diameter=3 mm). Scan rate=0.1 V/s. The peak potentials for the two-electron reduction of each lipid are shown.

FIG. 9 shows various permutations of lipids of the present invention.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

The present invention features redox-sensitive lipids developed as responsive liposomal carriers that can deliver anti-cancer ingredients to tumor tissues. One skilled in the art will appreciate that redox-sensitive liposomes may be useful in other situations including, but not limited to, packaging, carrying, or encapsulating nucleic acids or other biologically-active agents, transformation or transfection of eukaryotic or prokaryotic cells (“lipofection”), transport of liposomal contents across the blood-brain barrier, delivery of reagents in microfluidic devices and biological microelectromechanical systems, biological cell imaging/tomography, synthesis of nanostructures, and environmental remediation of soils and liquids. The redox-sensitive liposomes of the present invention are structurally optimized to preferentially accumulate in cancer tissues (preconcentration via enhanced permeability retention—EPR—effect) and to respond to high reductase activities (localized release facilitated by complementary redox potentials). Although disulfide-based liposomes that respond to redox stimulus/thiolysis do exist, these liposomes fail to respond to the reductases of cancer tissues. Indeed, the reductases of cancer tissues have no established reduction activities toward disulfide groups. Accordingly, the present invention provides the first liposomes capable of responding to the reductases of cancer tissues.

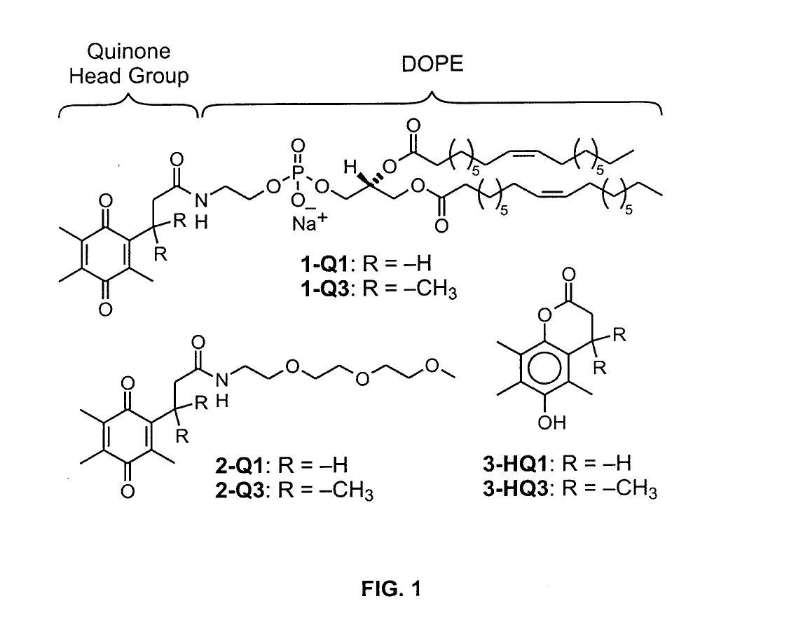

To depart from a disulfide-based strategy and create a new class of redox-responsive liposomal carriers that are structurally and electrochemically optimized for the delivery and enzymatically-triggered release of anti-cancer drugs to cancer tissues, it is desirable to create a liposome comprising trimethyl-locked quinone lipids that require a two-electron reductive activation to liberate the liposomal payload. The integration of a trimethyl-lock quinone switch within the liposome is critical because such a quinone switch has measurable activities toward several quinone reductases that are upregulated in cancer tissues. Because liposome-encapsulated drugs retain the pharmacokinetic properties of the carriers—meaning that the drugs are not pharmacologically active until released from the liposomes—it is widely perceived that triggered release of the active ingredients is necessary for the rapid delivery of anti-cancer drugs. In addition, triggered release is expected to be useful for overcoming the drawbacks associated with rapid clearance of liposomes from the blood by cells of the reticuloendothelial system. Thus, the development of new methods for the stimuli-triggered release of liposomal payloads is extremely important. The present disclosure shows that unilamellar (L_{α}) liposomes comprised of dioleoyl phosphatidylethanolamine (DOPE) lipids having a trimethyl-locked quinone (Q3) head group (1-Q3 in FIG. 1) liberate their contents upon reduction of Q3.

The following examples are offered by way of illustration and not by way of limitation.

As used herein, “Q” and “HQ” refer to the oxidized and reduced forms of the quinone, respectively, regardless of whether “HQ” is generated in situ (e.g., 1-HQ3) or synthesized (e.g., 3-HQ1). For example, 1-HQ1 is the reduced form of 1-Q1.

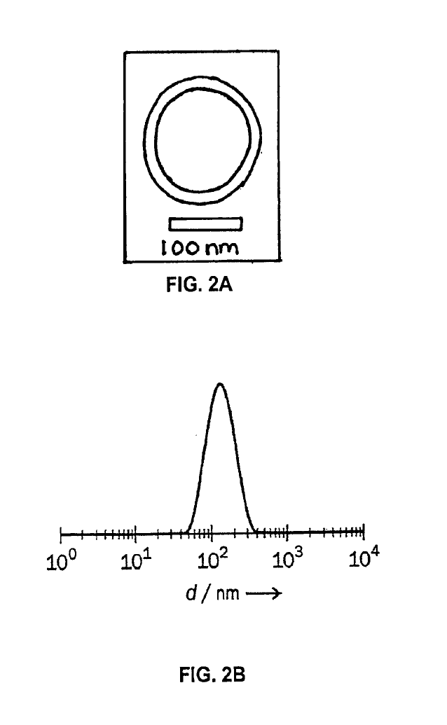

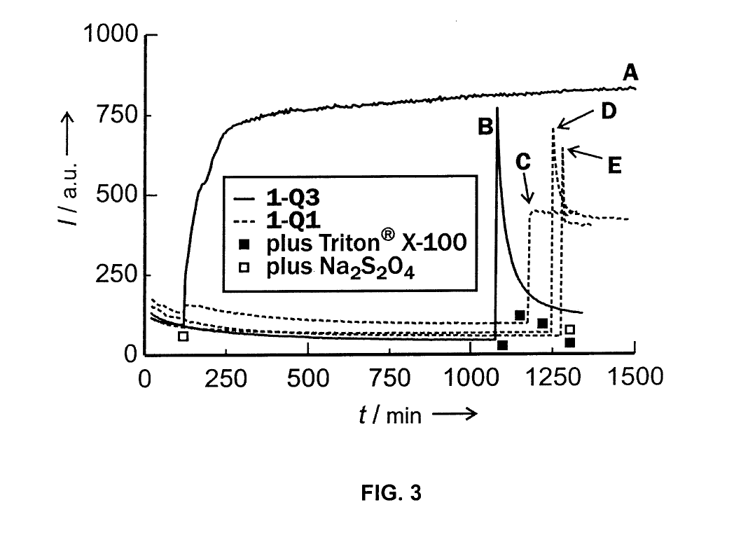

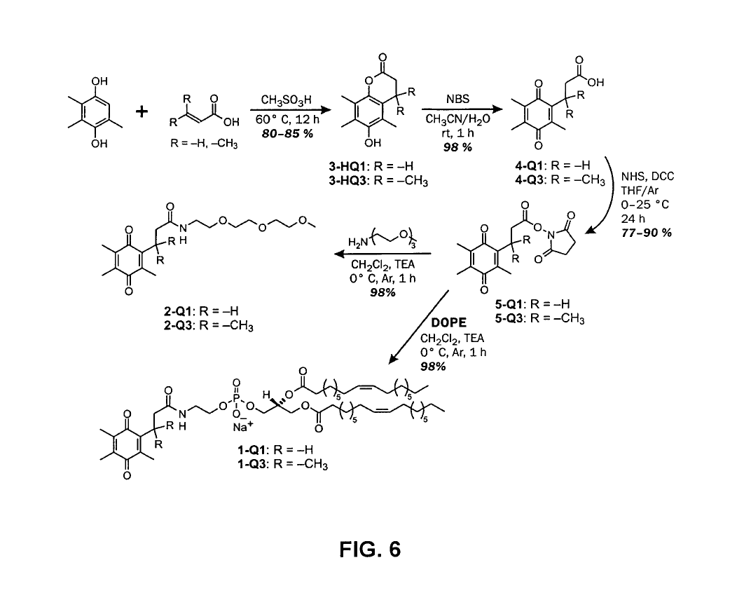

Quinone phospholipids 1-Q1 and 1-Q3 and the model derivatives 2-Q1 and 2-Q3 in FIG. 1 were synthesized by condensing the appropriate amines with the N-hydroxysuccinimide (NHS) esters of the quinine acids, NHS-Qx, x=1 or 3 (FIG. 6). The precursors of NHS-Q1 were synthesized using strategies that paralleled the preparation of NHS-Q3. All compounds yielded the predicted spectra by ¹H and ¹³C NMR spectrometry and ESI-TOF or GC mass spectrometry. Liposomes of 1-Q1 and 1-Q3 were prepared in pH 7.1/0.1 M phosphate buffer/0.1 M KCl using the extrusion technique, as described in EXAMPLE 1. Cryo-transmission electron microscopy (cryo-TEM) and dynamic light scattering (DLS) experiments reveal that 1-Q1 and 1-Q3 liposomes possess diameters in the 80-200 nm range (representative data in FIGS. 2A & 2B). When the liposomes were preloaded with calcein dyes (5×10⁻² M), the resulting DLS intensity distributions were indistinguishable from those of the calcein-free liposomes, indicating that the presence of dyes does not lead to significant changes in aggregate morphology. The quenched emission from quiescent, aerobic solutions of calcein-loaded liposomes over a 2-day period at room temperature reflects the stability of the liposomes under ambient conditions (see FIG. 3). As expected, and as shown in FIG. 3, addition of excess Triton X-100® detergent to the solutions of calcein-loaded liposomes caused release of the encapsulated dye, as evidenced by the rapid increase in emission intensity resulting from the dilution of calcein (FIG. 3, trace B for 1-Q3 and trace D for 1-Q1).

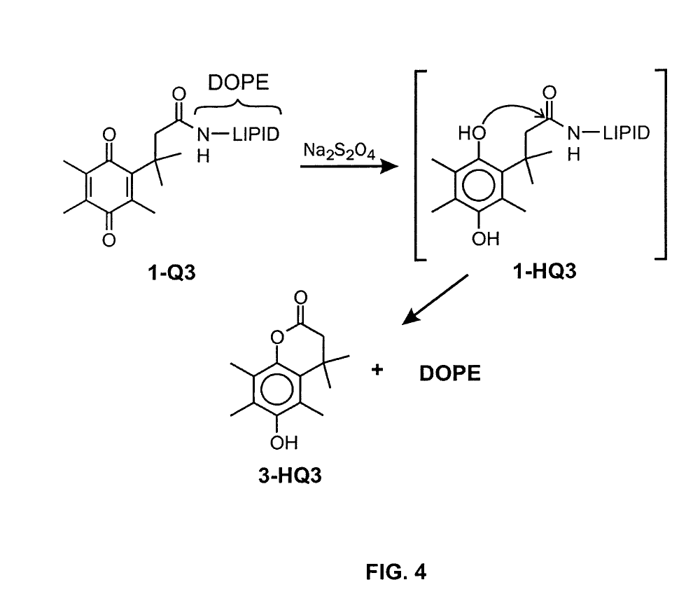

The two-electron reduction of various Q3 systems by chemical agents (e.g., Na₂S₂O₄ or NaBH₄) or by electrolysis produces the corresponding hydroquinone, HQ3, possessing a sterically congested configuration. HQ3 spontaneously dissociates from the parent structure as the lactone 3-HQ3. See, e.g., Ong W. & McCarley R. L. Macromolecules 2006; 39:7295-7301; Ong W. & McCarley R. L. Chem. Commun. 2005; 4699-4701. See also, Borchardt E. T. et al., J. Am. Chem. Soc. 1972; 94(26):9166-74. Based on time-course data that exhibit an immediate increase in fluorescence intensity of calcein following the addition of Na₂S₂O₄ to the calcein-loaded liposomes (FIG. 3, trace A), chemical reduction of liposomal 1-Q3 to 1-HQ3 resulted in the liberation of encapsulated calcein from the carrier. This outcome is consistent with the dissociation of in situ-generated 1-HQ3-containing liposomes into lactone 3-HQ3 and DOPE micelles (FIG. 4).

Unmodified DOPE is known to self-organize into inverted hexagonal columnar (H₁₁) micelles at pH 7 due to the head:tail volume ratio of this lipid. On the other hand, N-acylated DOPE lipids, such as 1-Q3 and 1-Q1, have larger head:tail volume ratios than DOPE and can be made to readily form liposomes at pH 7. Therefore, as depicted in FIG. 4, the structural transition of lamellar (L_{α})/liposomal 1-Q3 to micellar (H_{II}) DOPE (i.e., 1-Q3 (L_{α})→DOPE (H_{II})+3-HQ3) was accompanied by the release of calcein dyes. It is reasonable to assume that the H_{II} micelles are incapable of encapsulating calcein as efficiently as the lamellar liposomes. Thus, the inevitable expulsion of the entrapped dyes during the L_{α}→H_{II} structural transition is expected. The results described herein are the first demonstration of cargo unloading from a non-disulfide-based liposome that is triggered by reduction. These results will add to the repertoire of methods for masking non-liposome-forming phospholipids, including DOPE, with a liposome-forming, stimuli-sensitive N-substituent to induce liposome formation, such as those based on light, thiolysis and pH stimuli. Furthermore, the potential for this system to be responsive to reductase enzymes is high.

To assess the role of the L_{α}-to-H_{II} structural transition in the liberation of the dyes, liposomal 1-Q1 was employed as a control lipid because it lacks the geminal methyl (—CH₃) groups that are required to satisfy the trimethyl-lock configuration. Such a geometric requirement is a prerequisite for fast lactonization (t_{1/2}≦few hours). As a result, 1-HQ1 should not lactonize within the timescale of the dye-liberation experiments (see below). Thus, when the calcein-loaded liposomes of control lipid 1-Q1 were reduced, the dyes remained encapsulated inside the liposomes (fluctuation of trace C in FIG. 3 at 120 min) and addition of Triton® X-100 was required to release the dyes (trace C of FIG. 3 at 1150 min). This observation is in sharp contrast with liposomal 1-Q3, where the dyes were immediately liberated following the addition of Na₂S₂O₄ (compare traces A and C of FIG. 3 at 120 min). The inability of Na₂S₂O₄ to liberate the dyes from liposomal 1-Q1 suggests remarkably that the lamellar structure was maintained after reduction and that the permeability of the lipid barrier was unaffected by the redox conversion of 1-Q1 to 1-HQ1. These phenomena are unexpected, considering that previous accounts have demonstrated the dramatic effect of redox conversion on the stability of vesicles and micelles (Abbott N. L. et al., J. Am. Chem. Soc. 2005; 127:11576-77; Kakizawa Y. et al., Langmuir, 2001; 17:8044-48; Muñoz S. et al., J. Am. Chem. Soc. 1993; 115:4899-4900; Saji T. et al., J. Am. Chem. Soc. 1985; 107:6865-68). The ability of liposomal 1-HQ1 to retain calcein further suggests that the release of calcein from liposomal 1-HQ3 was not caused merely by the redox conversion of the Q3 head group to HQ3, but was rather imparted by the covalent disconnection of HQ3 from DOPE. The observation that the emission intensities of the liberated dyes remained similar regardless of the lysing agent used (compare, in FIG. 3, traces A with B, and C with D and control E) indicates that the release of dyes was nearly quantitative in all cases. It is unclear why free calcein quenches faster in the combined presence of Na₂S₂O₄ and Triton® X-100 (FIG. 3, control Curve E) relative to just Triton® X-100 (FIG. 3, curves B and D). Percent release of calcein was estimated using the well-known equation: % release=[(I−I₀)/(Iₘₐₓ−I₀)]×100, where I and Iₘₐₓ are the emission intensities after the addition of Na₂S₂O₄ and Triton® X-100, respectively, and h is the initial emission intensity.

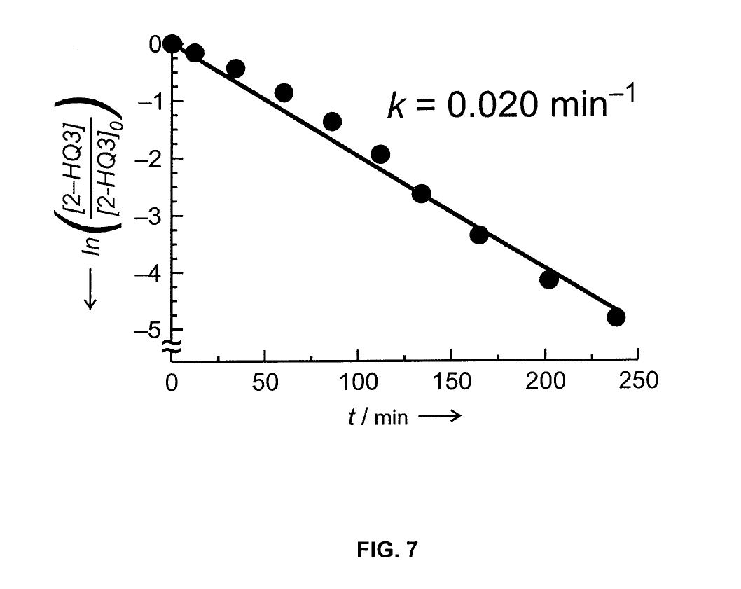

The dormancy of 1-HQ1 towards lactonization was verified by employing compounds 2-Q1 and 2-Q3 in aqueous milieu to model the relative reactivity, or lack thereof, of 1-HQ1 and 1-HQ3, respectively. Kinetic data recorded using ¹H NMR spectroscopy (see, e.g., Ong W. & McCarley R. L. Chem. Commun. 2005; 4699-4701) revealed that 2-HQ3, generated in situ by the reduction of 2-Q3 using Na₂S₂O₄, dissociated into lactone 3-HQ3 and 2-[2-(2-methoxy-ethoxy)-ethoxy]-ethylamine following first-order kinetics (k —0.02 min⁻¹, t_{1/2}˜30 min; FIG. 7). The experimental details of FIG. 7 follow. To a deoxygenated pD=7, 0.1M deutero-phosphate/0.1M KCl D₂O solution of the corresponding quinone-lipid in an NMR tube was added solid Na₂S₂O₄ (10-fold molar excess). Upon mixing of the Na₂S₂O₄ and the lipid solution inside the NMR tube, the yellow color of the solution was quenched instantly (completion <1 s). After the sample tube was loaded in an NMR spectrometer (Bruker DPX-400) for spectral acquisition, a macro was used to automatically acquire successive spectra at exactly 1 minute intervals. Each spectrum was set to acquire 2 scans with pre-scan delay; acquisition time and line broadening values were set to 2 s, 2 s and 1 Hz, respectively.

In contrast, reduced 2-HQ1, also generated in situ by the reduction of 2-Q1, remained stable over a 48-hr period. In other words, within the detection limits of ¹H NMR spectroscopy, release of lactone 3-HQ1 from 2-HQ1 was not observed. Therefore, it is indeed reasonable to assume that 1-HQ1 did not lactonize within the 1500-minute window of the dye-liberation experiments in FIG. 3.

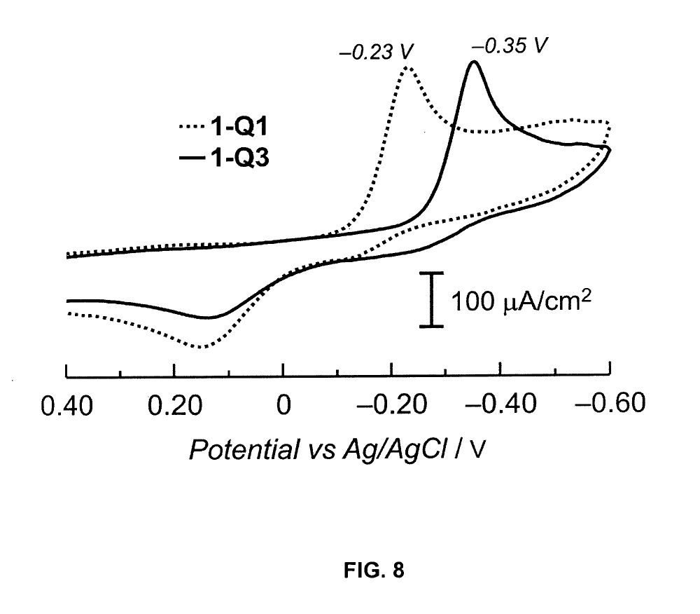

Voltammetric experiments were conducted on the model compounds 2-Q1 and 2-Q3 to test whether Na₂S₂O₄ is capable of reducing lipid 1-Q1 as effectively as 1-Q3. Voltammetric data (FIG. 8) reveal that two-electron reduction of 2-Q1 is thermodynamically easier than 2-Q3 by +0.12 V (i.e., ΔEₚ=E_{red} (2-Q1)−E_{red} (2-Q3)=+0.12 V), which translates to a Δ(ΔG)=5.5 kcal/mol. Therefore, assuming that the ΔEₚ value of the model compounds is similar to the ΔEₚ gap between lipids 1-Q1 and 1-Q3 (i.e., E_{red} (1-Q3)−E_{red} (2-Q3)˜E_{red} (1-Q1)−E_{red} (2-Q1)), it is reasonable to conclude that Na₂S₂O₄ reduces 1-Q1 to the same extent as 1-Q3. If anything, the aforementioned 5.5 kcal/mol gap suggests that reduction of 1-Q1 is thermodynamically easier than 1-Q3.

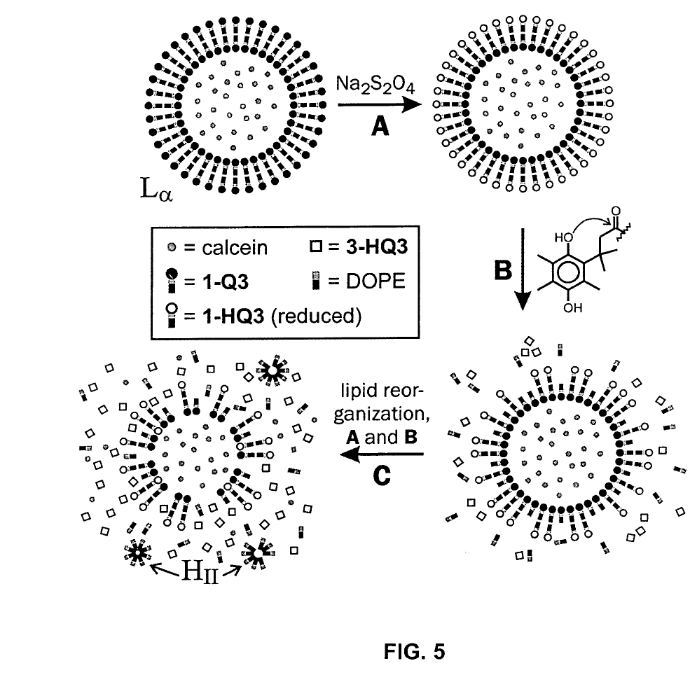

FIG. 5 outlines a mechanism for the sequence of reduction and lipid translocation events leading to the release of calcein dyes from liposomal 1-Q3. The addition of Na₂S₂O₄ reduces the 1-Q3 lipids that are located in the outer layer of the liposome to 1-HQ3 (FIG. 5, step A). Based on the results gathered from control experiments that demonstrate the unperturbed fluorescence intensity of calcein following the reduction of 1-Q1 to 1-HQ1 (trace C in FIG. 3, at 120 min), it is evident that the calcein dyes remained encapsulated even though the outer lipid layer of the liposomes was already reduced to 1-HQ3. Following reduction, and as described in FIG. 4, 1-HQ3 dissociates into lactone 3-HQ3 and DOPE (FIG. 5, Step B). As the 1-HQ3→DOPE+3-HQ3 dissociation process continues, the increasing concentration of liberated DOPE eventually exceeds the critical value for inverted micelle (H_{II}) formation, thereby allowing the calcein dyes to escape to the external solution. At this point, it is possible for Na₂S₂O₄ to access the other regions of the lipid bilayer and reduce the remaining 1-Q3 units. Although the undissociated lipids can still reorganize to restore the bilayer structure (FIG. 5, step C), either by transbilayer “flip-flopping,” lateral “squeezing,” or intervesicular migration, the resulting liposomes, now smaller in volume, will continue to be reduced (FIG. 5, steps A-C) until 1-Q3 is quantitatively converted to DOPE and 3-HQ3.

In conclusion, the present disclosure demonstrates that destruction of liposomal 1-Q3 occurs upon the reduction of the Q3 head groups by Na₂S₂O₄ to yield an intermediate which rapidly forms 3-HQ3 and DOPE, the latter being unable to support or maintain liposome formation at pH 7. The system presented here is applicable to drug-delivery systems that employ bioreductive activation of liposomal anti-cancer drugs, as most cancer tissues contain overexpressed quinone reductases.

Synthesis and characterization of 1-Q1, 1-Q3, 2-Q1, 2-Q3 and 3-Q1, voltammetry of 2-Q1 and 2-Q3, and the kinetics of reductive lactonization of 2-Q3 are described in detail below.

### Example 1

### Vesicle Preparation

1-Q1 or 1-Q3 (5-7 mg) was dissolved in CHCl₃ (10 mL) in a 25-mL round-bottom flask, and the lipid solution was evaporated to a thin lipid film using a rotary evaporator. The films were dried under vacuum for 1 hour and redissolved in pH 7.1/0.1 M phosphate buffer/0.1 M KCl at a concentration of 1 mg/mL. The solution was aged (one hour) with occasional vortexing (ca. 10-15 seconds at 20-minute intervals), after which it was freeze-thawed in a dry ice/acetone bath (7 times), followed by extrusion (12 times) at ambient temperature through two stacked, 100-nm pore Whatman Nuclepore polycarbonate track-etched membranes using a Lipex lipid extruder (Northern Lipids, Vancouver, BC, Canada). The buffer solutions used to prepare liposomes subjected to DLS measurements were filtered through a Whatman Anotop 20-nm membrane. For the preparation of calcein-loaded liposomes, the entire “extrusion” procedure described above was used, except that the buffered solvent also contained 5×10⁻² M dissolved calcein (Sigma-Aldrich, Milwaukee, Wis., USA). Following extrusion, the non-encapsulated dye were separated from the liposome-encapsulated dye by gel filtration (2 times) of the extruded solution through a column of Sephadex G-50 resin (GE Healthcare BioSciences, Piscataway, N.J., USA).

### Example 2

### Calcein-Release Experiments

The purified calcein-encapsulated liposomes were diluted with buffer to achieve an arbitrary concentration in the 10 pg/mL range (based on lipid). This concentration was determined spectrophotometrically using ε_{495, free}=ε_{495,encapsulated}=7.0×10⁴ M⁻¹ cm⁻¹ for calcein and an estimated bilayer thickness=4 nm, cross-sectional area of lipids=0.7 nm² and [encapsulated calcein]=5×10⁻² M. Solid Na₂S₂O₄ (85%, Sigma-Aldrich) or/and an appropriate aliquot of 7% (w/v) Triton X-100 (Sigma-Aldrich) were added to the cuvettes to attain the concentrations described in FIG. 3. Fluorescence intensities were recorded using a Perkin Elmer LS 50 luminescence spectrophotometer.

### Example 3

### DLS Measurements

Backscatter intensity (173°, 633-nm red laser) measurements were conducted at 25° C. on calcein-free and calcein-loaded liposomes using a Zetasizer Nano ZS (Malvern Instruments, Worcestershire, UK) particle-size analyzer.

### Example 4

### Cryo-Transmission Electron Microscopy

The method of vitrification of frozen hydrated samples was used. Buffered liposomal solution (2.5 μL) was deposited on a 400-mesh Cu grid covered with holey carbon film (Quantifoil Micro Tools EmbH, Jena Germany) and suspended in a Vitrobot (FEI, Hillsboro Oreg.). The grid was blotted for 2 seconds with filter paper, then the sample was vitrified using liquified ethane. A JEOL 2010F transmission electron microscope (JEOL Ltd., Tokyo, Japan) was used to image the specimens at 200 kV using a low-dose method, with the Gatan Model 626 cryo-stage (Gatan, Pleasanton Calif.) maintained at 92° K during the entire experiment. Images were recorded with a Gatan 4 k×4 k CCD camera (Gatan, Pleasanton Calif.) and processed with custom-built EMAN software (Baylor College of Medicine, Houston, Tex.).

### Example 5

### Synthesis of 3-HQ1,4-Q1, 5-Q1, 3-HQ3, 4-Q3 and 5-Q3

As depicted schematically in FIG. 6, 3-HQ3, 4-Q3 and 5-Q3 were synthesized as reported (J. Org. Chem., 1989, 54, 3303-3310). Their Q1 analogues (3-HQ1,4-Q1 and 5-Q1) were prepared using identical procedures, except as noted below. 3-HQ1: reaction time was extended to 14 hours. For purification, the concentrated solution of the crude product in ethyl acetate was diluted with n-hexanes, then refrigerated overnight to produce the purified product as a brown precipitate. Yield=46%; ¹H NMR (CDCl₃, 300 MHz) δ 4.53 (bs, 1H), 2.92 (t, 2H), 2.72 (t, 2H), 2.22 (s, 3H), 2.19 (s, 3H), 2.18 (s, 3H); ¹³C NMR (CDCl₃, 75 MHz) δ 169.58, 148.43, 144.45, 123.12, 121.99, 119.47, 118.24, 29.27, 21.51, 12.34, 12.21, 11.90; MS (GC), m/z: 206 (M⁺). 4-Q1: reaction time was shortened to 5 mins and temperature was 0° C. The crude product was recrystallized in CH₂Cl₂/Et₂O/n-hexanes to produce the product as dark-yellow crystals. Yield=88%; ¹H NMR (CDCl₃, 300 MHz) δ 2.82 (t, 2H), 2.51 (t, 2H), 2.07 (s, 3H), 2.02 (s, 6H); ¹³C NMR (CDCl₃, 75 MHz) δ 187.59, 187.00, 178.81, 141.93, 141.67, 140.87, 140.71, 32.77, 22.21, 12.54, 12.43, 12.37; MS (ESI) m/z: 223 ((M+H)⁺). 5-Q1: Yield=84%; ¹H NMR (CDCl₃, 300 MHz) δ 2.89 (m, 2H), 2.83 (m, 6H), 2.07 (s, 3H), 2.02 (s, 6H); ¹³C NMR (CDCl₃, 75 MHz) δ 187.38, 186.86, 169.17, 167.87, 142.39, 140.98, 140.66, 29.74, 25.69, 22.18, 12.54, 12.51, 12.40.

### Example 6

### Synthesis of 2-Q1 and 2-Q3

As depicted schematically in FIG. 6, 2-[2-(2-methoxy-ethoxy)-ethoxy]-ethylamine was synthesized as reported (J. Am. Chem. Soc., 2001, 123, 6536-6542). To 20 mL of a cooled (0° C.) solution of this amine (1.31 mmol) in dry CH₂Cl₂ was added triethylamine (3.9 mmol), then 5Qx (x=1 or 3, 1.31 mmol), and the reaction was allowed to reach to completion (1 hour) while maintained under argon atmosphere. The crude reaction mixture was concentrated, loaded to a SiO₂ column, and eluted using 7:1 ethyl acetate/methanol. R_{f} values of 2-Q1 and 2-Q3 in SiO₂ plate are ca. 0.75-0.80. The combined fractions were evaporated to dryness, and dried under vacuum to yield the products as yellow, viscous oils. 2-Q1: Yield=98%; ¹H NMR (CDCl₃, 300 MHz) δ 6.24 (bs, 1H), 3.65 (m, 6H), 3.56 (m, 4H), 3.43 (t, 2H), 3.38 (s, 3H), 2.81 (t, 2H), 2.31 (t, 2H), 2.07 (s, 3H), 2.01 (s, 6H); ¹³C NMR (CDCl₃, 75 MHz) δ 187.65, 187.21, 171.76, 142.74, 141.41, 140.75, 140.53, 71.99, 70.58, 70.53, 70.26, 69.91, 59.09, 39.36, 35.08, 23.17, 12.52, 12.41, 12.31; MS (ESI) m/z: 368.2 ((M+H)⁺). 2-Q3: Yield=98%; ¹H NMR (CDCl₃, 300 MHz) δ 6.07 (bs, 1H), 3.66-3.57 (m, 8H), 3.49 (t, 2H), 3.40 (s, 3H), 3.35 (m, 2H), 2.82 (s, 2H), 2.12 (s, 3H), 1.98 (s, 3H), 1.95 (s, 3H), 1.42 (s, 6H); ¹³C NMR (CDCl₃, 75 MHz) δ 191.35, 187.74, 172.06, 153.93, 143.92, 137.84, 137.40, 72.09, 70.61, 70.33, 70.04, 59.18, 49.16, 39.10, 38.37, 28.89, 14.23, 12.91, 12.26; MS (ESI) m/z: 396.2 ((M+H)⁺).

### Example 7

### Synthesis of 1-Q1 and 1-Q3

As depicted schematically in FIG. 6, the reaction conditions and reagent ratios were identical to the preparation of 2-Qx (x=1 or 3), except that the reaction times were extended to 4-6 hrs. For purification, the reaction mixture was diluted with CH₂Cl₂ to 50 mL, then extracted with 5% NaHCO₃ (1×50 mL). The organic layer was dried with Na₂SO₄, concentrated and loaded to a SiO₂ column, then eluted via a gradient of 1:1 CH₂Cl₂/EtOAc (to elute any unreacted 2-Qx, if any) followed by 3:1:2 CH₂Cl₂/MeOH/n-hexanes. The combined fractions were evaporated to dryness, and dried under vacuum to yield the products as yellow waxes. 1-Q1: Yield: 75%; ¹H NMR (CDCl₃, 400 MHz) δ 7.38 (bs, 1H), 5.33 (m, 4H), 5.22 (m, 1H), 4.36 (m, 1H), 4.14 (m, 1H), 3.95 (m, 4H), 3.49 (m, 2H), 2.76 (t, 2H), 2.33 (t, 2H), 2.26 (m, 4H), 2.03-1.96 (m, 17H), 1.54 (m, 4H), 1.26 (m, 40H), 0.88 (t, 6H); ¹³C NMR (CDCl₃, 75 MHz) δ 187.50, 187.33, 173.93, 173.68, 142.84, 141.54, 140.91, 140.53, 130.19, 129.80, 70.77, 64.94, 64.06, 63.17, 40.49, 34.78, 34.41, 34.24, 32.10, 29.98, 29.74, 29.52, 27.42, 25.13, 25.03, 22.88, 14.31, 12.57, 12.46, 12.31; MS (ESI) m/z: 946.6 ((M+H)⁴). 1-Q3: Yield: 96%; ¹H NMR (CDCl₃, 400 MHz) δ 7.65 (bs, 1H), 5.34 (m, 5H), 4.42 (m, 1H), 4.16 (m, 1H), 3.93 (m, 2H), 3.85 (m, 2H), 3.38 (m, 2H), 2.84 (s, 2H), 2.29 (m, 4H), 2.10 (s, 3H), 1.99 (m, 8H), 1.94 (s, 6H), 1.57 (m, 4H), 1.37 (s, 6H), 1.28 (m, 40H), 0.88 (t, 6H); ¹³C NMR (CDCl₃, 75 MHz) δ 191.49, 187.60, 174.15, 173.93, 172.86, 154.02, 143.73, 138.04, 137.54, 130.25, 129.75, 70.74, 65.21, 63.83, 63.07, 48.67, 39.84, 38.10, 34.50, 34.31, 32.10, 29.97, 29.74, 29.53, 28.84, 27.42, 25.08, 22.88, 14.32, 12.92, 12.29; MS (ESI) m/z: 946.6 (M).

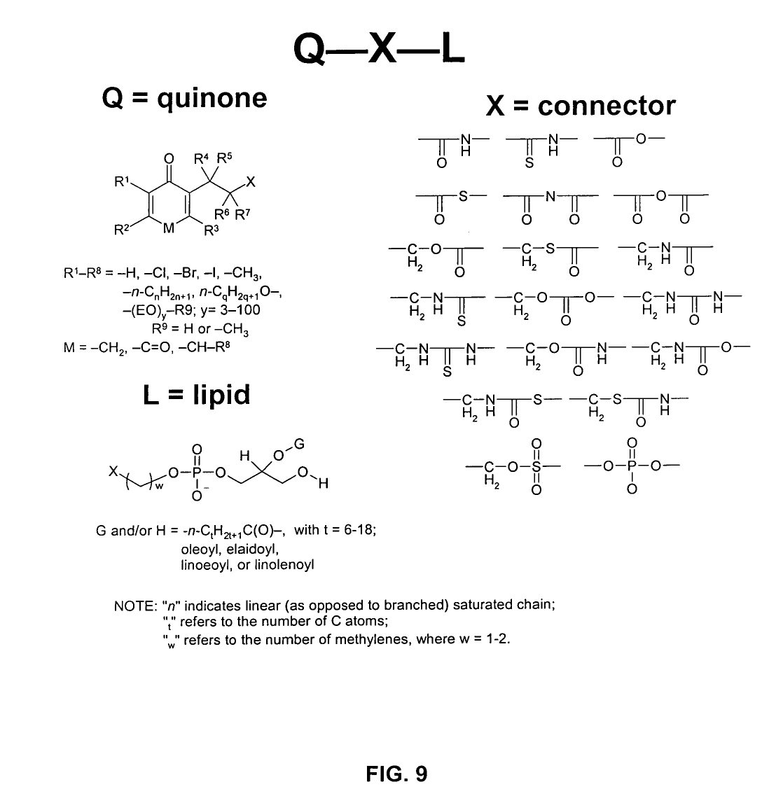

One of ordinary skill in the relevant art will appreciate that the liposomes of the present invention may be made using a single lipid of Formula I, or various mixtures of the lipids of Formula I (see FIG. 9). One of ordinary skill will also appreciate that while DOPE-quinones 1-Q1 and 1-Q3 are preferred for certain characteristics they possess, the lipids of the present invention (and the resulting liposomes of the present invention) may be modified in accordance with Formula I and FIG. 9 to produce other redox-gated lipids and liposomes with characteristics tailored to particular chosen applications.

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A lipid compound of the formula 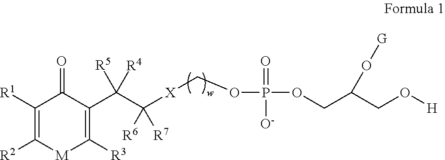
wherein:
R¹, R², R³, R⁴, R⁵, R⁶, R⁷, and R⁸ represent, independently, H, Cl, Br, I, CH₃, n-C_{y}H_{2y+1} (where y is an integer value from 1 to 3), n-C₁H₂ⱼ₊₁O (where j is an integer value from 1 to 3), or (EO)_{z}—R⁹ (where EO is ethylene oxide and z is an integer value from 3 to 100);
R⁹ is H, CH₃, or CF₃CH₂OC(O)CH₂;
M is CH₂, —C(O)—, or CH—R⁸;
X is —C(O)NH—, —C(S)NH—, —C(O)O—, —C(O)S—, —C(O)NHC(O)—, —C(O)OC(O)—, —CH₂OC(O)—, —CH₂SC(O)—, —CH₂NHC(O)—, —CH₂NHC(S)—, —CH₂OC(O)O—, —CH₂NHC(O)NH—, —CH₂NHC(S)NH—, —CH₂OC(O)—, —CH₂OC(O)NH—, —CH₂NHC(O)O—,
—CH₂NHC(O)S—, —CH₂SC(O)NH—, —CH₂OS(O)(O)—, or —CH₂OP(O)(O)O—; and
G and H are, independently, oleoyl, elaidoyl, linoeoyl, linolenoyl, or —C(O)[n-CₜH₂ₜ₊₁] (where t is an integer value from 6 to 18); and
w is an integer value between 1 and 2, indicating the number of methylenes (—CH₂—).

2. The lipid of claim 1, wherein R¹, R², R³, R⁴, and R⁵ are CH₃; R⁶ and R⁷ are H; M is —C(O)—; X is —C(O)NH—; w is 2; and both G and H are oleoyl.

3. A liposome comprising the lipid of claim 1.

4. A liposome comprising the lipid of claim 2.

5. The liposome of claim 3, further comprising one or more bioactive agents.

6. The liposome of claim 4, further comprising one or more bioactive agents.

7. A method of delivering one or more bioactive agents to cells, comprising encapsulating the agent in a liposome according to claim 4 to form a liposome-bioactive complex and contacting the cells with the complex.

8. The method of claim 7 wherein the bioactive agent is selected from the group consisting of antitumor agents, antibiotics, anthracycline antibiotics, immunodilators, anti-inflammatory drugs, and drugs acting on the central nervous system.

9. The method of claim 7, wherein the bioactive agent comprises a protein or a peptide.

10. The method of claim 7, wherein the bioactive agent comprises a nucleotide sequence.

11. A method of treating a disease in a patient comprising administering to the patient a therapeutically effective amount of a pharmaceutical composition containing one or more bioactive agents encapsulated in a liposome of claim 4.

12. A pharmaceutical formulation comprising the liposome of claim 6 and a physiologically-acceptable adjuvant thereof.

13. The formulation of claim 12 wherein the bioactive agent is selected from the group consisting of doxorubicin, daunorubicin, epirubicin, idarubicin, and mitoxantrone.
